(19) United States
(12) Patent Application Publication
(10) Pub. No.: US 2014/0066498 A1
(43) Pub. Date: Mar. 6, 2014

(54) TRITERPENOID DERIVATIVES, BENZENOID DERIVATIVES, AND PHARMACEUTICAL COMPOSITIONS CONTAINING THE SAME

(71) Applicant: National Cheng Kung University, Tainan City (TW)

(72) Inventor: TIAN-SHUNG WU, TAINAN CITY (TW)

(73) Assignee: National Cheng Kung University, Tainan City (TW)

(21) Appl. No.: 14/075,862

(22) Filed: Nov. 8, 2013

Related U.S. Application Data
(62) Division of application No. 13/067,230, filed on May 18, 2011.
(60) Provisional application No. 61/345,603, filed on May 18, 2010.
(60) Provisional application No. 61/345,606, filed on May 18, 2010.

(51) Int. Cl. C07D 493/04; C07D 317/64

(52) U.S. Cl. CPC C07D 493/04; C07D 317/64
USPC 514/456; 549/464; 514/464; 549/396

(57) ABSTRACT

The present invention relates to triterpenoid derivatives, benzenoid derivatives, and pharmaceutical compositions containing the same for treating cancers or inflammatory symptoms. According to the present invention, the triterpenoid derivatives and the benzenoid derivatives are respectively represented by the following formulas (I) and (II):

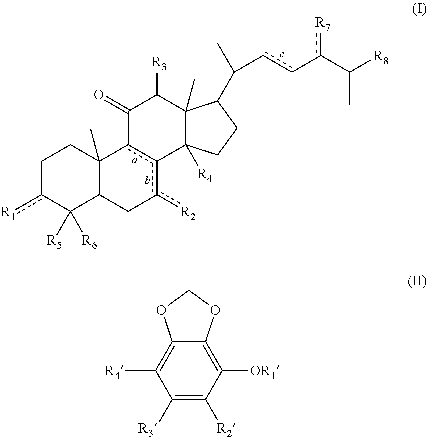

wherein, [symbol] R₁, [symbol] R₂, R₃, R₄, R₅, R₆, [symbol] R₇, R₈, [symbol] , [symbol] , [symbol] , R₁′, R₂′, R₃′, and R₄′ are defined the same as the specification.

### CROSS REFERENCE TO RELATED APPLICATION

This application claims priority to, and is a Divisional of, U.S. patent application Ser. No. 13/067,230, file on May 18, 2011, which claims the benefit of filing date of U.S. Provisional Application Ser. Nos. 61/345,603, and 61/345,606, respectively entitled “The Constituents and Biological Activities from the Fruiting Body of Taiwanofungus camphoratus”, and “Camphoratins and Derivatives as a New Class of Anticancer and Anti-inflammatory Agents”, both filed May 18, 2010 under 35 USC §119(e)(1).

### BACKGROUND OF THE INVENTION

1. Field of the Invention

The present invention relates to triterpenoid derivatives, benzenoid derivatives, and pharmaceutical compositions containing the same and, more particularly, to triterpenoid derivatives, benzenoid derivatives, and pharmaceutical compositions containing the same, which can be used as anticancer agents or anti-inflammatory agents.

2. Description of Related Art

Niuchangchih also named Taiwanofungus camphoratus (synonym: Ganoderma comphoratum; Antrodia cinnamomea; antrodia camphorata) (Polyporaceae, Aphyllophorales) is a rare and very precious medical fungus in Taiwan and is called as “national treasure of Taiwan”.

This microorganism, Taiwanofungus camphorates, is parasitic to the inner heart-wood wall of old hollow trunks of Cinnamomum kanehirai Hay. (Lauraceae). The growth rate of natural T. camphoratus in the wild is very slow, and it is difficult to cultivate in a greenhouse, making fruiting bodies expensive to obtain.

In traditional Taiwanese folk medicine, T. camphoratus has been used as an important health food for treating food, alcohol, and drug intoxication, diarrhea, abdominal pain, hypertension, itching, and liver cancer. It has been proven that T. camphoratus comprises a lot of active components, for example, polysaccharides such as β-glucan, triterpenoids, superoxide dismutase (SOD), adenosine, proteins including immune proteins, vitamins such as vitamin B and nicotinic acid, rare elements such as Ca, P and Ge, nucleic acid, lectine, amino acids, sterol, ligin, and antodia acid. These active components are considered having effects on anticancer, anti-allergen, anti-virus, anti-bacteria, and anti-hypertension. In addition, these active components can also be used to increase immune competency, inhibit platelet aggregation, decrease blood sugar and cholesterol, and protective the function of liver.

In addition, previous studies on the chemical constituents of the fruiting body of T. camphoratus also showed that this microorganism has a rich source of triterpenoidic acids, and some of which have shown anti-inflammatory, anticholinergic, and antiserotonergic activities. Furthermore, previous studies also showed that zhankuic acids A and C exhibited significant cytotoxicity against P-388 murine leukemia cells in vitro.

Although T. camphorates has a lot of active components for treating diseases, but it is uneasily available. Hence, if the active components can be isolated and further synthesized, it is possible to treat diseases with these isolated active components to increase the treatment effects.

### SUMMARY OF THE INVENTION

The object of the present invention is to provide triterpenoid derivatives and benzenoid derivatives, which are effective in treating cancers or inflammatory symptoms.

Another object of the present invention is to provide uses of triterpenoid derivatives or benzenoid derivatives, which can be served as anticancer agents or anti-inflammatory agents, and also used for manufacturing pharmaceutical compositions for treating cancer or inflammation.

A further object of the present invention is to provide pharmaceutical compositions for treating cancer, which comprise triterpenoid derivatives or benzenoid derivatives.

A further another object of the present invention is to provide a method for treating cancers or inflammatory symptoms by use of triterpenoid derivatives, benzenoid derivatives, or pharmaceutical compositions containing the same.

To achieve the object, the triterpenoid derivatives of the present invention are represented by the following formula (I):

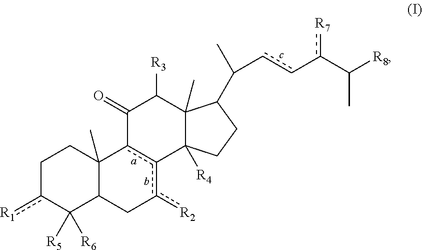

wherein,

### R₁ is —H, —OH, or ═O;

[symbol] R₂ is —H, —OH, or ═O, when [symbol] is a double bond, and [symbol] is a single bond; [symbol]
R₂ is —H, or —OH, when [symbol] is a single bond, and [symbol] is a double bond;
each of R₃, R₄, and R₅ independently is H, or OH;
R₆ is H, or C₁₋₆ alkyl; [symbol]
R₇ is —H, ═O, or —C₁₋₆ alkyl;
R₈ is C₁₋₆ alkyl, C₁₋₃ alkylol, C₁₋₃ carboxyl, or C₁₋₃ esteryl; and [symbol]
is a single bond, or a double bond.

According to the triterpenoid derivatives of the present invention, R₈ preferably is methyl, —(CH₂)—OH, —C(O)OH, or —C(O)OCH₃.

In addition, according to the triterpenoid derivatives of the present invention, R₆ may be H, or C₁₋₆ alkyl. Preferably, R₆ is H, or C₁₋₃ alkyl. More preferably, R₆ is H, methyl, ethyl, or propyl. Most preferably, R₆ is H, or methyl.

According to the triterpenoid derivatives of the present invention, [symbol] R₇ may be —H, ═O, or —C₁₋₆ alkyl. Preferably, [symbol] R₇ is —H, ═O, or —C₁₋₃ alkyl. More preferably, [symbol] R₇ is ═O, or methyl. Most preferably, [symbol] R₇ is ═O.

Furthermore, according to the triterpenoid derivatives of the present invention, R₈ may be C₁₋₆ alkyl, C₁₋₃ alkylol, C₁₋₃ carboxyl, or C₁₋₃ esteryl. Preferably, R₈ is C₁₋₃ alkyl, C₁₋₃ alkylol, C₁₋₃ carboxyl, or C₁₋₃ esteryl. More preferably, R₈ is methyl, —CH₂OH, —C(O)OH, or —C(O)OCH₃. Most preferably, R₈ is —C(O)OH, or —C(O)OCH₃.

In addition, when [symbol] is a double bond, [symbol] is a single bond; and when [symbol] is a single bond, [symbol] is a double bond. In addition, [symbol] may be a single bond or a double bond. Preferably, [symbol] is a single bond.

Preferably, [symbol] is a double bond, [symbol] is a single bond, and [symbol] is a single bond. In this case, [symbol] R₁ is —OH, or ═O, [symbol] R₂ is —H, —OH, and [symbol] R₇ is ═O, preferably. In addition, R₃ is H, R₄ is H, or OH, R₅ is H, R₆ is C₁₋₃ alkyl, and R₈ is —C(O)OH, or —C(O)OCH₃, preferably.

More specifically, the triterpenoid derivatives of the present invention is represented by the following formula (I-a) or (I-b):

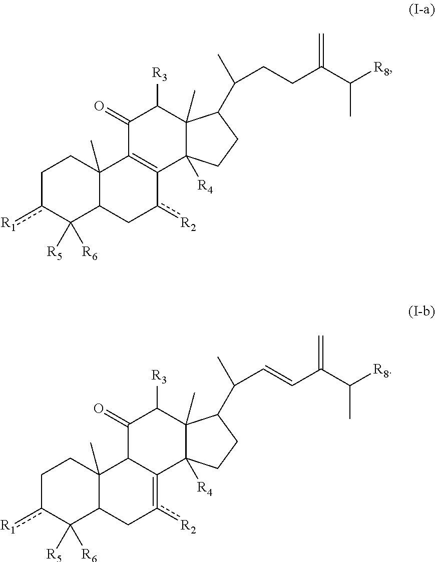

In the aforementioned formula (I-a) and (I-b), the substituted groups R₁ to R₈ are defined as the same in the formula (I). Furthermore, in the compounds represented by the formula (I), (I-a), or (I-b) of the present invention, the carboxylic acid moiety of the substituted group R₈ can be modified into a moiety selected from esters and amides with different functionalities. In addition, at least one of the hydroxyl groups in the compounds represented by the formula (I), (I-a), or (I-b) of the present invention can be modified into an ester or esters with different functionalities.

The specific examples of the aforementioned triterpenoid derivatives are the compounds represented by the following formula (I-1), (I-2), (I-3), (I-4), (I-5), (I-6), (I-7), (I-8), (I-9), or (I-10):

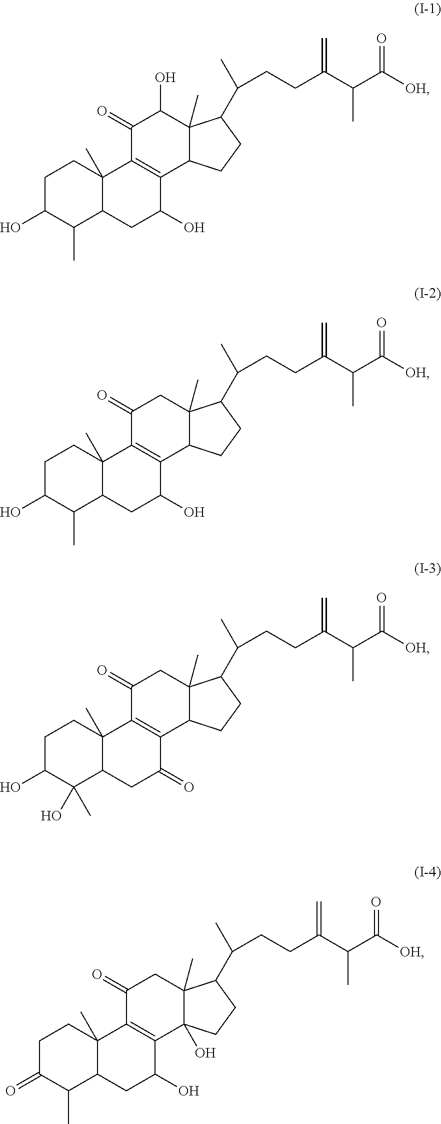 [SMILES: C=C(CCC(C)C1CCC2C3=C(C(=O)C(O)C21C)C1(C)CCC(O)C(C)C1CC3O)C(C)C(=O)O ; C=C(CCC(C)C1CCC2C3=C(C(=O)CC21C)C1(C)CCC(O)C(C)C1CC3O)C(C)C(=O)O ; C=C(CCC(C)C1CCC2C3=C(C(=O)CC21C)C1(C)CCC(O)C(C)(O)C1CC3=O)C(C)C(=O)O ; C=C(CCC(C)C1CCC2(O)C3=C(C(=O)CC12C)C1(C)CCC(=O)C(C)C1CC3O)C(C)C(=O)O]

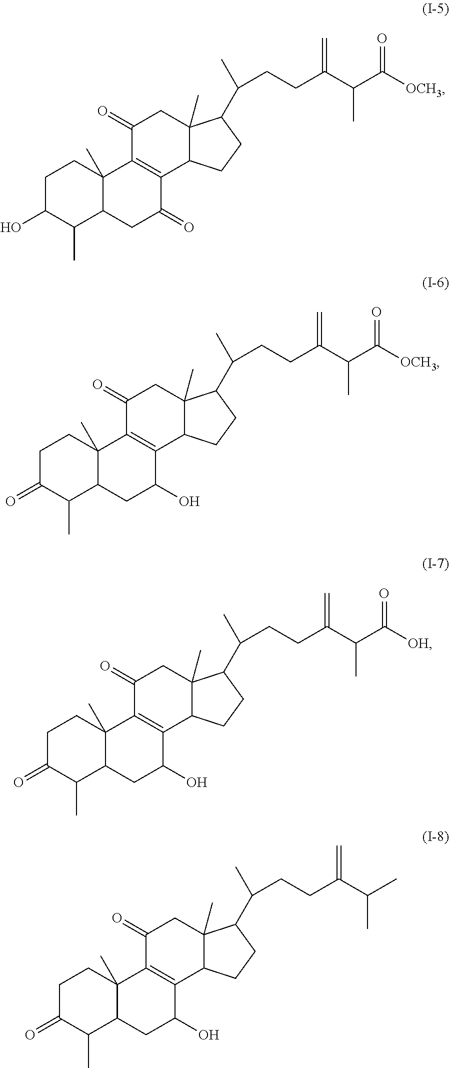 [SMILES: C=C(CCC(C)C1CCC2C3=C(C(=O)CC21C)C1(C)CCC(O)C(C)C1CC3=O)C(C)C(=O)OC ; C=C(CCC(C)C1CCC2C3=C(C(=O)CC21C)C1(C)CCC(=O)C(C)C1CC3O)C(C)C(=O)OC ; C=C(CCC(C)C1CCC2C3=C(C(=O)CC21C)C1(C)CCC(=O)C(C)C1CC3O)C(C)C(=O)O ; C=C(CCC(C)C1CCC2C3=C(C(=O)CC21C)C1(C)CCC(=O)C(C)C1CC3O)C(C)C]

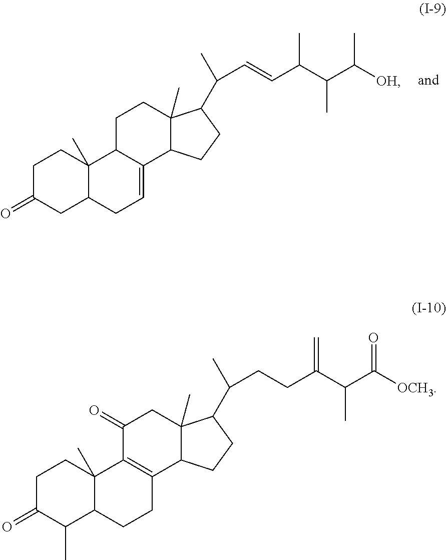 [SMILES: CC(O)C(C)C(C)/C=C/C(C)C1CCC2C3=CCC4CC(=O)CCC4(C)C3CCC21C ; C=C(CCC(C)C1CCC2C3=C(C(=O)CC21C)C1(C)CCC(=O)C(C)C1CC3)C(C)C(=O)OC]

The present invention also provides a use of the aforementioned triterpenoid derivatives as anticancer agents or anti-inflammatory agents. In addition, the present invention further provides a use of the aforementioned triterpenoid derivatives for manufacturing a pharmaceutical composition for treating cancer or inflammation. Therefore, the obtained pharmaceutical composition for treating cancer of the present invention comprises: an effective amount of the aforementioned triterpenoid derivatives, and a pharmaceutically acceptable carrier. Furthermore, the obtained pharmaceutical composition for treating inflammation of the present invention also comprises: an effective amount of the aforementioned triterpenoid derivatives, and a pharmaceutically acceptable carrier. Furthermore, the present invention provides a method for treating cancer or inflammation, which comprises the following steps: treating an object with the aforementioned pharmaceutical composition.

In addition, the present invention further provides an extract of T. camphorates, which comprises the aforementioned triterpenoid derivatives.

The present invention also provide benzenoid derivatives, which are represented by the following formula (II):

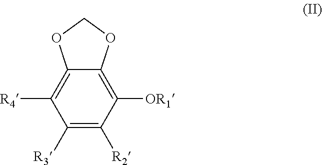

wherein,
R₁′ is C₁₋₆ alkyl;
R₂′ is C₁₋₆ alkyl, or C₁₋₆ alkoxy;
R₃′ is H, C₁₋₆ alkyl,

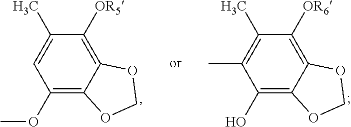

R₄′ is hydroxyl, C₁₋₆ alkoxy, or

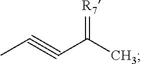

each of R₅′, and R₆′ independently is C₁₋₆ alkyl; and

### R₇′ is O, or CH₂.

According to the benzenoid derivatives of the present invention, R₁′ may be C₁₋₆ alkyl. Preferably, R₁′ is C₁₋₃ alkyl. More preferably, R₁′ is methyl, or ethyl. Most preferably, R₁′ is methyl.

In addition, according to the benzenoid derivatives of the present invention, R₂′ is C₁₋₆ alkyl, or C₁₋₆ alkoxy. Preferably, R₂′ is C₁₋₃ alkyl, or C₁₋₃ alkoxy. More preferably, R₂′ is methyl, or methoxy. Most preferably, R₁′ and R₂′ are methyl.

According to the benzenoid derivatives of the present invention, R₃′ may be H, C₁₋₆ alkyl,

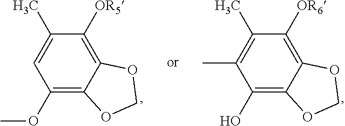

wherein R₅′, and R₆′ independently is C₁₋₆ alkyl. Preferably, R₃′ is H, C₁₋₃ alkyl,

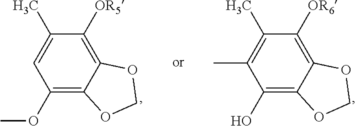

wherein R₅′, and R₆′ independently is C₁₋₃ alkyl. More preferably, R₃′ may be H, methyl,

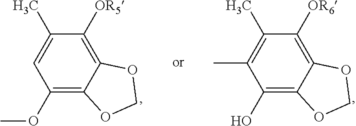

R₅′, and R₆′ independently is methyl.

Furthermore, according to the benzenoid derivatives of the present invention, R₄′ may be hydroxyl (—OH), C₁₋₆ alkoxy, or

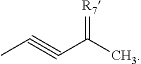

Preferably, R₄′ is hydroxyl, C₁₋₃ alkoxy, or

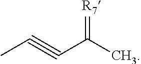

More preferably, R₄′ is

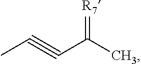

### and R₇′ is CH₂.

The specific examples of the aforementioned benezoid derivatives are the compound represented by the following formula (II-1), (II-2), (II-3), (II-4), or (II-5):

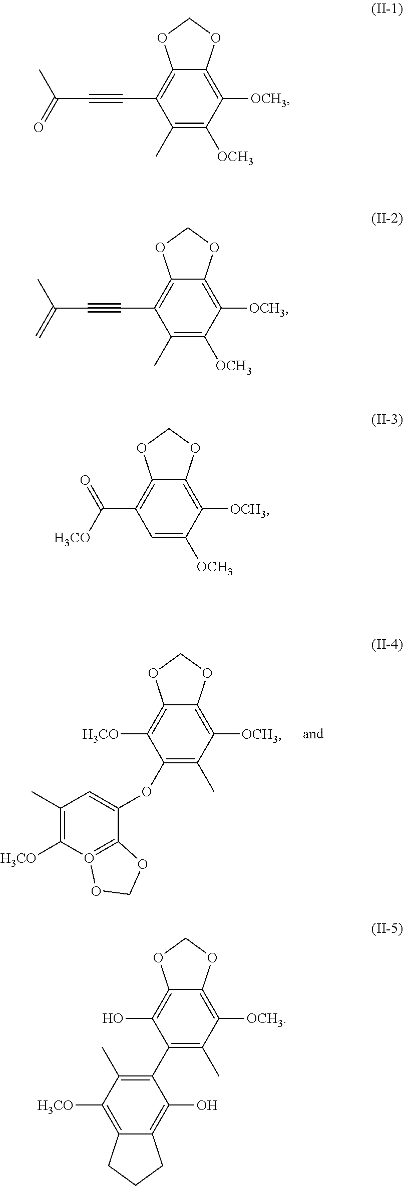

The present invention also provides a use of the aforementioned benezoid derivatives as anticancer agents or anti-inflammatory agents. In addition, the present invention further provides a use of the aforementioned benezoid derivatives for manufacturing a pharmaceutical composition for treating cancer or inflammation. Therefore, the obtained pharmaceutical composition for treating cancer of the present invention comprises: an effective amount of the aforementioned benezoid derivatives, and a pharmaceutically acceptable carrier. Furthermore, the obtained pharmaceutical composition for treating inflammation of the present invention also comprises: an effective amount of the aforementioned triterpenoid derivatives, and a pharmaceutically acceptable carrier. Furthermore, the present invention provides a method for treating cancer or inflammation, which comprises the following steps: treating an object with the aforementioned pharmaceutical composition.

In addition, the present invention further provides an extract of T. camphorates, which comprises the aforementioned benezoid derivatives.

According to the pharmaceutical composition of the present invention, “acceptable” means that the carrier must be compatible with the active ingredient such as triterpenoid and benzenoid (and preferably, capable of stabilizing the active ingredient) and not deleterious to the subject to be treated. Suitable carriers include microcrystalline cellulose, mannitol, glucose, defatted milk powder, polyvinylpyrrolidone, and starch, or a combination thereof.

In addition, the term “treating” used in the present invention refers to the application or administration of the pharmaceutical composition to a subject with cancer or inflammatory symptoms, in order to cure, heal, alleviate, relieve, alter, remedy, ameliorate, improve, or affect the disease.

Furthermore, “an effective amount” used herein refers to the amount of each active agent required to confer therapeutic effect on the subject. The effective amount may vary according to the route of administration, excipient usage, and co-usage with other active agents.

Other objects, advantages, and novel features of the invention will become more apparent from the following detailed description when taken in conjunction with the accompanying drawings.

### DETAILED DESCRIPTION OF THE PREFERRED EMBODIMENT

### Fungal Material

Wild fruiting bodies of T. camphoratus, which grew in Ping-Tung County, Taiwan, were purchased from the Kaohsiung Society for Wildlife and Nature in 2003. The fungus was identified by Dr. Tun-Tschu Chang (Taiwan Forestry Research Institute). A voucher specimen (TSWu 2003005) was deposited in the Department of Chemistry, National Cheng Kung University, Tainan, Taiwan.

### Embodiment 1

### Extraction and Isolation of Triterpenoid Derivatives

The fresh fruiting body of T. camphorates (1.0 kg) was extracted with EtOH four times (4×10 L) under reflux for 8 h. The EtOH extract was concentrated to afford brown syrup (161 g) and then partitioned between water and n-hexane. The n-hexane layer (9.3 g) was chromatographed on silica gel and eluted with EtOAc in n-hexane (0-100% of EtOAc, gradient) to obtain ten fractions. Fraction 4 was rechromatographed on a silica gel column using n-hexane-Me₂CO (19:1) as eluent to yield compounds I-8 (3.0 mg), I-9 (6.0 mg), I-10 (4.5 mg), I-19 (22.0 mg), I-20 (90.2 mg), I-21 (22.1 mg), and I-22 (16.5 mg). Compound I-22 (41.1 mg) was obtained in the same way from fraction 8. The water layer (145 g) was filtered and concentrated under reduced pressure to give a brown syrup (55 g) and a water-insoluble portion (89 g). The water-insoluble portion was chromatographed on a silica gel column using CHCl₃-MeOH mixtures of increasing polarity for elution to obtain ten fractions (WI-1-WI-10). Compounds I-1 (2.2 mg), I-5 (2.0 mg), I-6 (14.2 mg), I-9 (1.0 mg), I-14 (1.29 g), I-15 (53.8 mg), and I-21 (62.2 mg) were obtained from a combined fraction (fractions WI-1 and WI-2) by silica gel column chromatography with gradient elution (CHCl₃-Me₂CO, 39:1 to 14:1). Fraction WI-3 was separated on a silica gel column using i-Pr₂O-MeOH (19:1) as the eluent to yield compounds I-11 (141.5 mg), I-18 (11.0 mg), I-16 (122.9 mg), and I-12 (53.0 mg). Fraction WI-4 was chromatographed on a silica gel column with i-Pr₂O-MeOH (12:1) to give compounds I-7 (11.3 mg), I-18 (38.0 mg), I-16 (708.0 mg), and I-12 (66.5 mg). Compounds I-2 (5.0 mg), I-4 (2.2 mg), I-7 (3.4 mg), and I-13 (286.2 mg) were obtained from fraction WI-5 using silica gel column chromatography (eluent, CHCl₃-MeOH, 12:1). Fractions WI-6 and WI-7 were combined and rechromatographed on a silica gel column with CHCl₃-MeOH (6:1) as the mobile phase to afford compounds I-3 (3.8 mg) and I-13 (1.81 g). Compound I-17 (1.16 g) was isolated from a combined fraction (fractions WI-8 and WI-9) by silica gel column chromatography using i-Pr₂O-MeOH (4:1) as the eluent.

Melting points of the isolated compounds were determined on a Yanagimoto MP-S3 micro-melting point apparatus. IR spectra were recorded on a Shimazu FTIR spectrometer Prestige-21. Optical rotations were measured using a Jasco DIP-370 Polarimeter. UV spectra were obtained on a Hitachi UV-3210 spectrophotometer. ESI and HRESI mass spectra were recorded on a Bruker APEX II mass spectrometer. The NMR spectra, including ¹H NMR, ¹³C NMR, COSY, NOESY, HMBC, HMQC experiments, were recorded on Bruker AVANCE-500 and AMX-400. Silica gel (E. Merck 70-230, 230-400 mesh) was used for column chromatography.

### Compound I-1

### 3α,7β,11α-trihydroxy-11-oxo-4α-methylergosta-8,24(28)-dien-26-oic acid

The compound I-1 was isolated as colorless powders, and the analysis data thereof are listed as follow.

mp 117-119° C.; [α]_{D}²⁵+221 (c 0.001, MeOH); UV (MeOH) λₘₐₓ (log ε) 255 (3.49) nm; IR (KBr) νₘₐₓ 3408, 2959, 2930, 2875, 1709, 1660, 1215, 1059 cm⁻¹; ¹H NMR and ¹³C NMR, see the following Tables 1 and 2; ESIMS m/z 511 [M+Na]⁺; HRESIMS m/z 511.3038 (calculated for C₂₉H₄₄O₆Na 511.3035).

These data helped to establish the structure of the compound I-1, and the result showed that the structure of the compound I-1 is represented by the following formula (I-1):

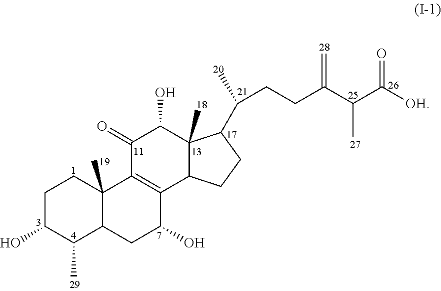 [SMILES: C=C(CC[C@@H](C)C1CCC2C3=C(C(=O)[C@H](O)[C@@]21C)[C@@]1(C)CC[C@@H](O)[C@@H](C)C1C[C@H]3O)C(C)C(=O)O]

### Compound I-2

### 3α,7β-dihydroxy-11-oxo-4α-methylergosta-8,24(28)-dien-26-oic acid

The compound I-2 was isolated as colorless syrup, and the analysis data thereof are listed as follow.

[α]_{D}²⁵+54 (c 0.006, MeOH); UV (MeOH) λₘₐₓ (log ε) 255 (3.79) nm; IR (KBr) νₘₐₓ 3420, 2962, 2935, 2878, 1709, 1659, 1217, 1083 cm⁻¹; ¹H NMR and ¹³C NMR, see the following Tables 1 and 2; ESIMS m/z 495 [M+Na]⁺; HRESIMS m/z 495.3089 (calculated for C₂₉H₄₄O₅Na 495.3086).

These data helped to establish the structure of the compound I-2, and the result showed that the structure of the compound I-2 is represented by the following formula (I-2):

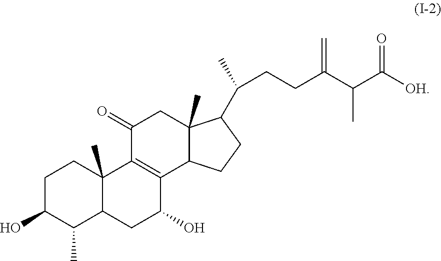 [SMILES: C=C(CC[C@@H](C)C1CCC2C3=C(C(=O)C[C@@]21C)[C@@]1(C)CC[C@H](O)[C@@H](C)C1C[C@H]3O)C(C)C(=O)O]

### Compound I-3

### 3α,4β-dihydroxy-7,11-dioxo-4α-methylergosta-8,24(28)-dien-26-oic acid

The compound I-3 was isolated as colorless powders, and the analysis data thereof are listed as follow.

mp 186-188° C.; [α]_{D}²⁵+57 (c 0.067, MeOH); UV (MeOH) λₘₐₓ (log ε) 271 (3.80) nm; IR (KBr) νₘₐₓ 3411, 2966, 2936, 2878, 1709, 1674, 1230, 1062 cm⁻¹; ¹H NMR and ¹³C NMR, see the following Tables 1 and 2; ESIMS m/z 509 [M+Na]; HRESIMS m/z 509.2874 (calculated for C₂₉H₄₂O₆Na 509.2879).

These data helped to establish the structure of the compound I-3, and the result showed that the structure of the compound I-3 is represented by the following formula (I-3):

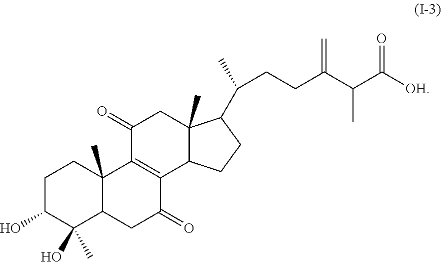 [SMILES: C=C(CC[C@@H](C)C1CCC2C3=C(C(=O)C[C@@]21C)[C@@]1(C)CC[C@@H](O)[C@](C)(O)C1CC3=O)C(C)C(=O)O]

### Compound I-4

### 7β,14α-dihydroxy-3,11-dioxo-4α-methylergosta-8,24(28)-dien-26-oic acid

The compound I-4 was isolated as colorless powders, and the analysis data thereof are listed as follow.

mp 175-177° C.; [α]_{D}²⁵+34° (c 0.004 MeOH); UV (MeOH) λₘₐₓ (log ε) 246 (3.97) nm; IR (KBr) νₘₐₓ 3444, 2971, 2936, 2878, 1708, 1670, 1229, 1187, 1068, cm⁻¹; ¹H NMR and ¹³C NMR, see the following Table 1 and 3; ESIMS m/z 509 [M+Na]⁺; HRESIMS m/z 509.2875 (calculated for C₂₉H₄₂O₆Na 509.2879).

These data helped to establish the structure of the compound I-4, and the result showed that the structure of the compound I-4 is represented by the following formula (I-4):

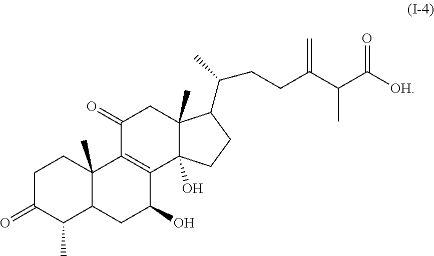 [SMILES: C=C(CC[C@@H](C)C1CC[C@@]2(O)C3=C(C(=O)C[C@]12C)[C@@]1(C)CCC(=O)[C@@H](C)C1C[C@@H]3O)C(C)C(=O)O]

### Compound I-5

### methyl-3α-hydroxy-7,11-dioxo-4α-methylergosta-8,24(28)-dien-26-oate

The compound I-5 was isolated as colorless syrup, and the analysis data thereof are listed as follow.

[α]_{D}²⁵+166 (c 0.007, MeOH); UV (MeOH) λₘₐₓ (loge) 260 (3.68) nm; IR (KBr) νₘₐₓ 3491, 2959, 2936, 2877, 1730, 1678, 1235, 1202, 1169 cm⁻¹; ¹H NMR and ¹³C NMR, see the following Tables 1 and 3; ESIMS m/z 507 [M+Na]⁺; HRESIMS m/z 507.3088 (calculated for C₃₀H₄₄O₅Na 507.3086).

These data helped to establish the structure of the compound I-5, and the result showed that the structure of the compound I-5 is represented by the following formula (I-5):

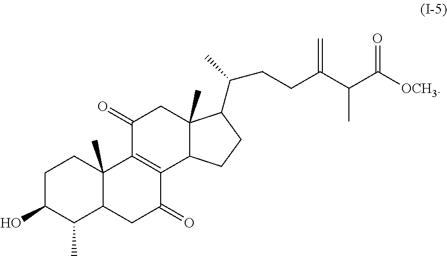 [SMILES: C=C(CC[C@@H](C)C1CCC2C3=C(C(=O)C[C@@]21C)[C@@]1(C)CC[C@H](O)[C@@H](C)C1CC3=O)C(C)C(=O)OC]

### Compound I-6

### methyl-7β-hydroxy-3,11-dioxo-4α-methylergosta-8,24(28)-dien-26-oate

The compound I-6 was isolated as colorless powders, and the analysis data thereof are listed as follow.

mp 100-101° C.; [α]_{D}²⁵+174 (c 0.008, MeOH); UV(MeOH) λₘₐₓ (log ε) 251 (4.05) nm; IR (KBr) νₘₐₓ 3386, 2967, 2877, 1732, 1711, 1669, 1235, 1197, 1167, 1083 cm⁻¹; ¹H NMR and ¹³C NMR, see the following Tables 1 and 3; ESIMS m/z 507 [M+Na]⁺; HRESIMS m/z 507.3083 (calculated for C30H44O5Na 507.3086).

These data helped to establish the structure of the compound I-6, and the result showed that the structure of the compound I-6 is represented by the following formula (I-6):

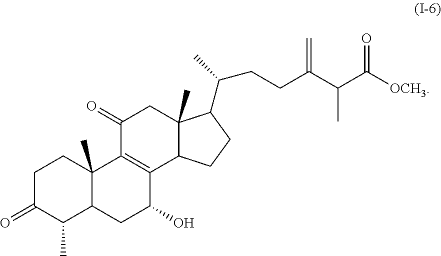 [SMILES: C=C(CC[C@@H](C)C1CCC2C3=C(C(=O)C[C@@]21C)[C@@]1(C)CCC(=O)[C@@H](C)C1C[C@H]3O)C(C)C(=O)OC]

### Compound I-7

### 7α-hydroxy-3,11-dioxo-4α-methylergosta-8,24(28)-dien-26-oic acid

The compound I-7 was isolated as colorless powders, and the analysis data thereof are listed as follow.

mp 196-198° C.; [α]_{D}²⁵+139 (c 0.007, MeOH); UV (MeOH) λₘₐₓ (log ε) 247 (4.33) nm; IR (KBr) νₘₐₓ 3420, 2964, 2930, 2875, 1707, 1659, 1171 cm⁻¹; ¹H NMR and ¹³C NMR, see the following Tables 1 and 3; ESIMS m/z 493 [M+Na]⁺; HRESIMS m/z 493.2929 (calculated for C₂₉H₄₂O₅Na 493.2930).

These data helped to establish the structure of the compound I-7, and the result showed that the structure of the compound I-7 is represented by the following formula (I-7):

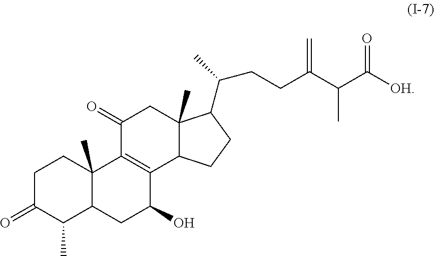 [SMILES: C=C(CC[C@@H](C)C1CCC2C3=C(C(=O)C[C@@]21C)[C@@]1(C)CCC(=O)[C@@H](C)C1C[C@@H]3O)C(C)C(=O)O]

### Compound I-8

### 4α-methylergosta-8,24(28)-dien-3,11-dione

The compound I-8 was isolated as colorless syrup, and the analysis data thereof are listed as follow.

[α]_{D}²⁵+41 (c 0.008, MeOH); UV (MeOH) λₘₐₓ (log ε) 248 (3.94) nm; IR(KBr) νₘₐₓ 2965, 2940, 2877, 1711, 1678 cm⁻¹; ¹H NMR and ¹³C NMR, see the following Tables 1 and 3; ESIMS m/z 447 [M+Na]⁺; HRESIMS m/z 447.3237 (calculated for C₂₉H₄₄O₂Na 447.3239).

These data helped to establish the structure of the compound I-8, and the result showed that the structure of the compound I-8 is represented by the following formula (I-8):

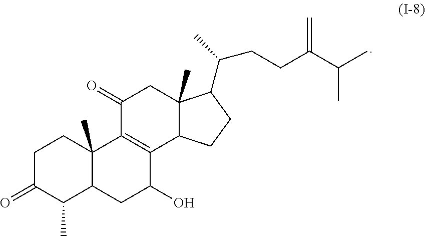 [SMILES: C=C(CC[C@@H](C)C1CCC2C3=C(C(=O)C[C@@]21C)[C@@]1(C)CCC(=O)[C@@H](C)C1CC3O)C(C)C]

### Compound I-9

### (25S)-26-hydroxy-ergosta-7,22-dien-3-one

The compound I-9 was isolated as colorless powders, and the analysis data thereof are listed as follow.

mp 192-193° C.; [α]_{D}²⁵+128 (c 0.003, MeOH); IR (KBr) λₘₐₓ 3336, 2956, 2873, 1716, 1024 cm⁻¹; ¹H NMR and ¹³C NMR, see the following Tables 1 and 3; ESIMS m/z 435 [M+Na]⁺; HRESIMS m/z 435.3242 (calculated for C₂₈H₄₄O₂Na 435.3239).

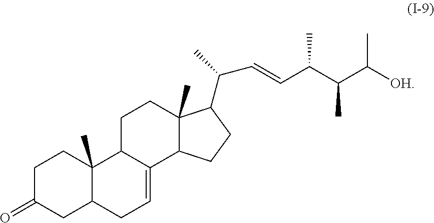 [SMILES: CC(O)[C@@H](C)[C@@H](C)/C=C/[C@@H](C)C1CCC2C3=CCC4CC(=O)CC[C@]4(C)C3CC[C@@]21C]

### Compound I-10

### methyl-3,11-dioxo-4α-methyl-14β-ergosta-8,24(28)-dien-26-oate

The compound I-10 was isolated as colorless powders, and the analysis data thereof are listed as follow.

mp 100-102° C.; [α]_{D}²⁵+164 (c 0.005, MeOH); UV (MeOH) λₘₐₓ (log ε) 250 (4.35) nm; IR (KBr) νₘₐₓ 2953, 2873, 2856, 1738, 1709, 1669, 1460, 1453, 1375, 1077 cm⁻¹; ¹H NMR and ¹³C NMR, see the following Tables 1 and 3; ESIMS m/z 491 [M+Na]⁺; HRESIMS m/z 491.3135 (calculated for C₃₀H₄₄O₄Na 491.3137).

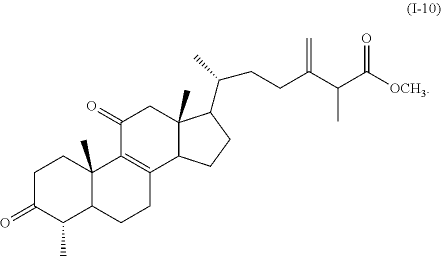 [SMILES: C=C(CC[C@@H](C)C1CCC2C3=C(C(=O)C[C@@]21C)[C@@]1(C)CCC(=O)[C@@H](C)C1CC3)C(C)C(=O)OC]

TABLE 1
¹³C NMR Spectroscopic Data for Compounds I-1-I-4 (in
pyridine-d5) and I-5-I-10 (in CDCl₃)

| Position | I-1^{a} | I-2^{a} | I-3^{a} | I-4^{b} | I-5^{a} | I-6^{a} | I-7^{b} | I-8^{a} | I-9^{a} | I-10^{a} |
|---|---|---|---|---|---|---|---|---|---|---|
| 1 | 29.7 | 29.8 | 28.8 | 36.3 | 27.8 | 35.7 | 34.6 | 35.5 | 38.8 | 35.1 |
| 2 | 30.6 | 30.7 | 26.6 | 38.0 | 29.1 | 37.8 | 37.5 | 37.0 | 38.1 | 37.8 |
| 3 | 70.3 | 70.2 | 74.3 | 211.0 | 70.3 | 212.3 | 212.8 | 213.7 | 211.9 | 213.1 |
| 4 | 35.0 | 35.4 | 74.0 | 43.8 | 34.5 | 43.8 | 43.8 | 44.8 | 44.2 | 44.3 |
| 5 | 40.4 | 40.4 | 44.6 | 47.7 | 41.1 | 48.2 | 44.6 | 51.0 | 42.9 | 50.6 |
| 6 | 32.9 | 32.7 | 37.0 | 35.0 | 38.1 | 32.5 | 31.3 | 21.3 | 30.0 | 21.1 |
| 7 | 70.1 | 70.1 | 203.5 | 70.5 | 202.1 | 69.9 | 70.2 | 30.6 | 117.1 | 32.3 |
| 8 | 154.3 | 155.1 | 155.0 | 154.3 | 144.7 | 153.2 | 153.0 | 157.5 | 139.4 | 154.6 |
| 9 | 141.2 | 143.0 | 144.2 | 141.2 | 153.7 | 141.2 | 140.7 | 139.1 | 48.9 | 138.0 |
| 10 | 37.7 | 38.1 | 40.4 | 38.5 | 38.7 | 37.0 | 37.2 | 38.2 | 34.4 | 36.4 |
| 11 | 202.8 | 201.8 | 203.0 | 199.5 | 203.1 | 201.3 | 200.9 | 200.3 | 21.7 | 200.4 |
| 12 | 81.7 | 58.9 | 58.0 | 49.5 | 57.5 | 57.9 | 57.6 | 58.1 | 39.3 | 53.2 |
| 13 | 50.7 | 48.2 | 47.7 | 47.5 | 47.3 | 47.6 | 47.1 | 47.6 | 43.3 | 44.0 |
| 14 | 47.3 | 53.9 | 49.9 | 83.2 | 49.5 | 53.0 | 51.2 | 53.5 | 55.0 | 55.3 |
| 15 | 25.4 | 25.6 | 25.7 | 32.1 | 24.9 | 24.8 | 23.1 | 24.1 | 22.9 | 29.3 |
| 16 | 27.8 | 28.4 | 28.2 | 26.2 | 27.8 | 27.8 | 27.5 | 28.0 | 28.1 | 30.4 |
| 17 | 46.0 | 55.0 | 54.3 | 49.5 | 53.9 | 54.4 | 55.1 | 55.8 | 55.8 | 55.8 |
| 18 | 12.4 | 12.7 | 12.3 | 16.6 | 11.9 | 12.1 | 12.2 | 12.1 | 12.1 | 22.3 |
| 19 | 18.3 | 17.1 | 19.7 | 17.1 | 15.9 | 17.5 | 16.3 | 17.8 | 12.4 | 18.0 |
| 20 | 36.5 | 36.4 | 36.1 | 35.7 | 35.7 | 35.7 | 35.8 | 36.3 | 40.5 | 33.3 |
| 21 | 18.3 | 18.8 | 18.8 | 19.4 | 18.5 | 18.5 | 18.4 | 18.8 | 21.1 | 19.5 |
| 22 | 34.9 | 34.7 | 34.6 | 34.0 | 33.8 | 33.9 | 33.8 | 34.8 | 136.7 | 32.8 |
| 23 | 32.1 | 31.9 | 31.9 | 32.1 | 31.2 | 31.2 | 30.7 | 31.3 | 130.4 | 31.9 |
| | | | | | 31.0^{c} | 31.0^{c} | | | | 31.8^{c} |
| 24 | 150.7 | 150.6 | 150.6 | 150.2 | 148.5 | 148.4 | 148.1 | 156.9 | 38.1 | 148.5 |
| 25 | 46.8 | 46.9 | 46.9 | 46.7 | 45.7 | 45.7 | 45.1 | 34.3 | 40.8 | 45.7 |
| | | | | | 45.5^{c} | 45.5^{c} | | | | 45.5^{c} |
| 26 | 176.9 | 177.0 | 177.0 | 176.7 | 175.0 | 175.0 | 177.4 | 22.2 | 66.9 | 175.0 |
| 27 | 17.3 | 17.2 | 17.3 | 17.2 | 16.4 | 16.4 | 16.2 | 22.3 | 12.7 | 16.4 |
| | | | | | 16.3^{c} | 16.3^{c} | | | | 16.3^{c} |
| 28 | 110.5 | 110.7 | 110.7 | 110.5 | 110.9 | 110.9 | 111.5 | 106.6 | 18.3 | 110.9 |
| 29 | 17.1 | 17.1 | 27.5 | 12.0 | 15.7 | 11.5 | 11.9 | 12.2 | | 11.6 |
| OMe | | | | | 51.9 | 51.9 | | | | 51.9 |

^{a}Recorded at 100 MHz at 25° C.
^{b}Recorded at 125 MHz at 25° C.
^{c}Chemical shifts for 25-epimer.

TABLE 2
¹H NMR Spectroscopic Data for Compounds I-1-I-4 (in
pyridine-d5)

| Position | I-1^{a} | I-2^{a} | I-3^{a} | I-4^{b} |
|---|---|---|---|---|
| 1 | 1.93 m | 1.85 m | 2.10 td (13.2, 3.2) | 1.50 m |
| | 2.78 m | 2.85 m | 3.04 dt (13.2, 3.2) | 3.28 m |
| 2 | 1.86 m | 1.86 m | 1.92 m | 2.40 m |
| | 1.93 m | 1.89 m | 2.74 m | 2.52 m |
| 3 | 3.89 d (1.6)^{c} | 3.91 d (2.4) | 4.02 br s | |
| 4 | 1.64 m | 1.62 m | | 2.39 m |
| 5 | 2.13 m | 2.02 m | 2.65 m | 1.50 m |
| 6 | 1.74 m | 1.67 m | 2.90 dd (13.2, | 2.23 m |
| | | | 3.2) | |
| | 2.42 m | 2.39 m | 3.14 t (13.2) | 2.51m |
| 7 | 4.52 t (8.4) | 4.50 t (8.4) | | 4.98 t (8.4) |
| 12 | 4.44 s | 2.43 d (13.2) | 2.46 d (13.2) | 2.74 d |
| | | | | (15.8) |
| | | 2.95 d (13.2) | 2.97 d (13.2) | 2.89 d |
| | | | | (15.8) |
| 14 | 3.57 dd | 2.66 dd (12.0, | 2.67 m | |
| | (12.0, 6.8) | 6.0) | | |
| 15 | 2.19 m | 2.01 m | 1.66 m | 1.80 m |
| | 2.50 m | 2.49 m | 2.74 m | |
| 16 | 1.42 m | 1.45 | 1.44 | 1.60 m |
| | | | | 1.83 m |
| 17 | 2.42 m | 1.43 m | 1.42 m | 1.75 m |
| 18 | 0.90 s | 0.88 s | 0.72 s | 1.22 s |
| 19 | 1.57 s | 1.49 s | 1.99 s | 1.45 s |
| 20 | 1.41 m | 1.40 m | 1.38 | 1.56 m |
| 21 | 1.11 d (7.6) | 0.89 d (7.6) | 0.87 d (5.2) | 1.01 d (6.5) |
| 22 | 1.37 m | 1.31 m | 1.30 m | 1.27 m |
| | 1.81 m | 1.75 m | 1.75 m | 1.88 m |
| . . . | 2.25 m | 2.20 m | 2.20 m | 2.23 m |
| | 2.44 m | 2.39 m | 2.38 m | 2.42 m |
| 25 | 3.45 q (6.8) | 3.45 q (7.2) | 3.45 q (7.2) | 3.44 q (7.2) |
| 27 | 1.48 d (7.2) | 1.49 d (6.8) | 1.49 d (7.2) | 1.47 d (7.2) |
| 28 | 5.07 s | 5.06 s | 5.06 s | 5.06 s |
| | 5.23 s | 5.22 s | 5.23 s | 5.21 s |
| 29 | 1.18 d (6.8) | 1.18 d (6.4) | 1.61 s | 1.11 d (6.6) |

^{a}Recorded at 400 MHz at 25° C.
^{b}Recorded at 500 MHz at 25° C.
^{c}J values (in Hz) in parentheses.

TABLE 3
¹H NMR Spectroscopic Data for Compounds I-5-I-10 (in CDCl₃)

| Position | I-5^{a} | I-6^{a} | I-7^{b} | I-8^{a} | I-9^{a} | I-10^{a} |
|---|---|---|---|---|---|---|
| 1 | 1.40 m | 1.25 m | 1.26 m | 1.33 m | 1.49m | 1.33 m |
| | 2.50 m | 2.95 m | 2.95 m | 3.18 m | 2.13 m | 2.88 m |
| 2 | 1.72 m | 2.35 m | 2.40 m | 2.35 m | 2.30 m | 2.37 m |
| | 1.94 m | 2.49 m | 2.49 m | 2.51 m | 2.42 td (14.4, 8.8) | 2.50 m |
| 3 | 3.79 br s | | | | | |
| 4 | 1.74 m | 2.35 m | 2.40 m | 2.36 m | 2.24 m | 2.38 m |
| 5 | 2.12 m | 1.39 m | 1.46 m | 1.39 m | 1.83 m | 1.41 m |
| 6 | 2.25 t (15.1)^{c} | 1.56 m | 1.57 m | 1.43 m | 1.27 m | 1.42 m |
| | 2.41 dd (15.1, 3.0) | 2.49 m | 1.89 m | 1.78 m | 1.83 m | 1.78 m |
| 7 | | 4.39 t (8.0) | 4.26 d (2.0) | 2.18 m | 5.18 br s | 2.11 m |
| | | | | 2.37 m | | 2.30 m |
| 9 | | | | | 1.75 m | |
| 11 | | | | | 1.54 m | |
| 11 | | | | | 1.64 m | |
| 12 | 2.40 d (13.6) | 2.32 d (14.0) | 2.38 d (14.5) | 2.33 d (14.4) | 1.27 m | 2.18 d (13.9) |
| 12 | 2.89 d (13.6) | 2.83 d (14.0) | 2.84 d (14.5) | 2.80 d (14.4) | 2.04 m | 2.47 d (13.9) |
| 14 | 2.62 dd (12.4, 7.0) | 2.71 m | 2.78 m | 2.64 dd (12.0, 7.6) | 1.81 m | 2.09 m |
| 15 | 1.47 m | 1.90 m | 1.90 m | 1.52 m | 1.41 m | 1.37 m |
| | 2.55 m | 2.09 m | 2.07 m | 1.81 m | 1.52 m | 1.93 m |
| 16 | 1.25 m | 1.44 m | 1.40 m | 1.42 m | 1.29 m | 1.47 m |
| | 1.98 m | 1.96 m | 1.90 m | 1.81 m | 1.73 m | 2.08 m |
| 17 | 1.42 m | 1.38 m | 1.46 m | 1.48 m | 1.28 m | 1.39 m |
| 18 | 0.67 s | 0.77 s | 0.72 s | 0.74 s | 0.57 s | 1.02 s |
| 19 | 1.31 s | 1.44 s | 1.26 s | 1.34 s | 1.01 s | 1.37 s |
| 20 | 1.42 m | 1.41 m | 1.43 m | 1.47 m | 2.05 m | 1.46 m |
| 21 | 0.93 d (5.6) | 0.92 d (5.5) | 0.93 d (6.0) | 0.95 d (5.6) | 1.02 d (7.2) | 0.90 d (6.4) |
| 22 | 1.18 m | 1.25 m | 1.32 m | 1.22 m | 5.25 m | 1.17 m |
| | 1.57 m | 1.58 m | 1.59 m | 1.53 m | | 1.47 m |
| 23 | 1.95 m | 1.98 m | 2.00 m | 1.89 m | 5.25 m | 1.95 m |
| | 2.16 m | 2.15 m | 2.17 m | 2.10 m | | 2.14 m |
| 24 | | | | | 2.23 m | |
| 25 | 3.13 q (7.0) | 3.12 q (6.8) | 3.16 q (7.0) | 2.24 m | 1.58 m | 3.13 q (7.0) |
| 26 | | | | 1.06 d (6.8) | 3.45 dd (10.4, 6.4) | |
| | | | | | 3.56 dd (10.4, 6.4) | |
| 27 | 1.28 d (7.0) | 1.27 d (6.8) | 1.30 d (7.5) | 1.03 d (6.8) | 0.86 d (6.8) | 1.28 d (7.0) |
| 28 | 4.92 s, 4.88 s | 4.91 s, 4.89 s | 4.94 s | 4.67 s | 1.00 d (6.8) | 4.92 s, 4.88 s |
| | 4.90 s, 4.86 | 4.87 s, 4.85 | 4.99 s | 4.74 s | | 4.90 s, 4.87 |
| | s^{d} | s^{d} | | | | s^{d} |
| 29 | 0.96 d (6.4) | | 1.03 d (7.0) | 1.29 d (6.8) | | 1.04 d (6.6) |
| OMe | 3.66 s | 3.66 s | | | | 3.66 s |

^{a}Recorded at 400 MHz at 25° C.
^{b}Recorded at 500 MHz at 25° C.
^{c}J values (in Hz) in parentheses.
^{d}Chemical shifts for 25-epimer.

### Compounds I-11 to I-22

Other compounds obtained from Embodiment 1 are known compounds, including zhankuic acids A-C (I-11-I-13), zhankuic acid A methyl ester (I-14), antcin A (I-15), antcin C (I-16), antcin K (I-17), methyl antcinate H (I-18), eburicol (I-19), ergosterol D (I-20), methyl 4α-methylergost-8,24(28)-dien-3,11-dion-26-oate (I-21), and ergosterol peroxide (I-22).

### Cytotoxicity Assay

Compounds I-1-I-19 were assayed for cytotoxic activity against KB (human cancer cell), and KB-YIN (multidrug-resistant strain) in vitro.

The results of the cytotoxicity assay are shown in the following Table 4.

TABLE 4

EC₅₀ (μM)

| Compound | KB | KB-VIN |
|---|---|---|
| I-1 | NA@20 | NA@20 |
| I-2 | 1.8 | NA@20 |
| I-3 | 0.3 | 2.3 |
| I-4 | 1.0 | 1.4 |
| I-5 | 0.45 | 2.7 |
| I-6 | 2.0 | 2.9 |
| I-7 | 15.0 | 17.5 |
| I-8 | NA@20 | NA@20 |
| I-9 | NA@20 | NA@20 |
| I-10 | NA@20 | NA@20 |
| I-11 | 3.0 | 6.2 |
| I-12 | 7.3 | 8.5 |
| I-13 | 15.5 | 6.4 |
| I-14 | >20 (21) | >20 (25) |
| I-15 | 4.9 | 10.0 |
| I-16 | NA@20 | NA@20 |
| I-17 | NA@20 | NA@20 |
| I-18 | NA@20 | NA@20 |
| I-19 | >20 (34) | >20 (18) |

Many of the compounds, including I-2-I-7, I-11-I-13, and I-15, showed moderate to potent cytotoxic activity with EC₅₀ values ranging from 0.3 to 15.5 μM. Among them, compounds I-3 and I-5 showed the best cytotoxicity against KB cell line with EC₅₀ values of 0.3 and 0.45 μM, respectively. Compounds I-4 and I-6 also showed potent cytotoxicity against KB with EC₅₀ of 1.0 and 2.0 μM, respectively. More importantly, compounds I-4 and I-6 retained their activity against multi-resistant strain KB-VIN with EC₅₀ of 1.4 and 2.9 μM, respectively.

In addition, the anti-inflammatory activities of compounds I-2, I-6, I-9, and I-10-I-22 were evaluated by examining their effects on LPS-induced iNOS-dependent NO production and NOX-dependent ROS production in murine microglial cells (BV2) and peripheral human neutrophils (PMN). The processes for these assays are shown as follow. Microglial cell culture and measurements of mitric oxide (NO). The murine microglial cell line (BV2) was cultured, and the production of NO was measured by the methods as previously described (Wang, Y. H.; Wang, W. Y.; Chang, C. C.; Liou, K. T.; Sung, Y. J.; Liao, J. F.; Chen, C. F.; Chang, S.; Hou, Y. C.; Chou, Y. C.; Shen, Y. C. J. Biomed. Sci. 2006, 13, 127-141).

### Measurement of NADPH Oxidase (NOX) Activity

NADPH oxidase activity was measured as previously described (Wang, Y. H.; Wang, W. Y.; Chang, C. C.; Liou, K. T.; Sung, Y. J.; Liao, J. F.; Chen, C. F.; Chang, S.; Hou, Y. C.; Chou, Y. C.; Shen, Y. C. J. Biomed. Sci. 2006, 13, 127-141).

### Measurement of 1,1-Diphenyl-2-Picrylhydrazyl (DPPH) Radical-Scavenging Capacity

DPPH radical-scavenging capacity assay was performed as previously report (Lin, L. C.; Wang, Y. H.; Hou, Y. C.; Chang, S.; Liou, K. T.; Chou, Y. C.; Wang, W. Y.; Shen, Y. C. J. Pharm. Pharmacol. 2006, 58, 129-135).

The results are listed in the following Table 5.

TABLE 5
Summary of the effects of compounds I-2, I-6, I-9, and I-10-I-22
on NADPH oxidase (NOX) activity^{a} in murine microglial cells (BV2) and
peripheral human neutrophils (PMN) and nitric oxide synthase (NOS)
activity^{b} in murine microglial cells

| | IC₅₀ (μM) in NOX | IC₅₀ (μM) in NOX | |
|---|---|---|---|
| | activity from BV2 | fMLP-induced NOX | IC₅₀ (μM) in |
| | cell lysate | activation in PMN | NOS |
| I-2 | N.A. | 32.1 ± 3.5* | 15.7 ± 0.9* |
| I-6 | N.A. | 11.2 ± 2.3* | 2.5 ± 0.6* |
| I-9 | N.A. | 17.5 ± 3.9* | 12.7 ± 2.2* |
| I-10 | N.A. | 15.8 ± 4.0* | 1.6 ± 0.6* |
| I-11 | N.A. | 22.1 ± 6.7* | 3.6 ± 0.8* |
| I-12 | N.A. | N.A. | 9.6 ± 0.7* |
| I-13 | 40.3 ± 3.5* | N.A. | 16.2 ± 0.9* |
| I-14 | N.A. | 8.4 ± 2.1* | 0.6 ± 0.3* |
| I-15 | 45.9 ± 7.9* | 29.2 ± 6.7* | 4.1 ± 0.5* |
| I-16 | N.A. | 22.6 ± 3.3* | 4.2 ± 1.2* |
| I-17 | N.A. | 47.2 ± 8.4* | N.A. |
| I-18 | 16.0 ± 8.1* | 18.1 ± 5.9* | 2.5 ± 0.3* |
| I-19 | N.A. | 21.9 ± 6.3* | 22.3 ± 2.9* |
| I-20 | N.A. | 27.9 ± 5.6* | 30.6 ± 0.8* |
| I-21 | N.A. | 16.2 ± 4.3* | 1.5 ± 0.7* |
| I-22 | N.A. | 20.3 ± 6.4* | 6.3 ± 1.8* |
| DPI | 0.4 ± 0.2 | 0.3 ± 0.1 | — |
| L-NAME | — | — | 25.8 ± 2.5 |

^{a}NADPH oxidase (NOX) activity were measured as reactive oxygen species production by triggering with NADPH (200 μM) or fMLP (2 μM) in the presence 1-50 μM of test drugs in BV2 cell lysate or peripheral human neutrophils (PMN). Diphenyleneiodonium (DPI, a NOX inhibitor) was included as a positive control for NOX inhibition.
^{b}NO production was measured in the presence of 1-50 μM of test drugs.
L-NAME (a non-selective NOS inhibitor) was included a positive control.
Data were calculated as 50% inhibitory concentration (IC₅₀) and expressed as the mean ± S.E.M. from 3-6 experiments performed on different days using BV2 cell lysate or PMN from different passages or donors.
N.A.: not active.
“—”: samples not tested.
*P < 0.05 as compared with relative positive control.

Compounds I-6, I-10, I-11, I-14-I-16, I-18, and I-21 significantly inhibited NOS activity with IC₅₀ values of 2.5, 1.6, 3.6, 0.6, 4.1, 4.2, 2.5, and 1.5 μM, respectively. These compounds were more potent than L-NAME (IC₅₀ 25.8 μM), a nonspecific NOS inhibitor, at inhibiting LPS-induced NO production, as shown in Table 5. The remaining compounds, except for compound I-20, effectively inhibited NOS activity with IC₅₀ values ranging from 6.3 to 22.3 μM.

In addition, NOX is the major ROS-producing enzyme in activated inflammatory cells. The previous report has shown that drugs with anti-inflammatory activity also show potent NOX-inhibitory action. The data for evaluating the effects of these compounds on NOX activity in lysates of microglial cells and PMN suggest that none of the tested compounds were potent inhibitors of NOX in lysates of microglial cells and PMN, relative to the specific NOX inhibitor DPI (IC₅₀ 0.4 and 0.3 μM, respectively), as shown in Table 5. In addition, the free radical-scavenging capacities of these compounds were examined in a cell-free DPPH solution. None of these tested compounds showed significant free radical-scavenging activity.

In many circumstances, inflammation orchestrates the microenvironment around tumors, contributing to proliferation, survival and migration. Cancer cells also use selectins, chemokines, and their receptors (involved in inflammatory response) for invasion, migration and metastasis. Thus, the triterpenoid derivatives of the present invention with both potent cytotoxicity and anti-inflammatory activity have a great potential to be developed into anti-inflammatory drugs for the treatment of NO-dependent neurodegenerative disorders, anticancer drugs, or anticancer agents producing synergistic effects with current anticancer drugs.

### Embodiment 2

### Extraction and Isolation of Benzenoid Derivatives

The fresh fruiting body of T. camphorates (1.0 kg) was extracted with EtOH four times (4×10 L) under reflux. The EtOH extract was concentrated to afford brown syrup (161 g) and then partitioned between MeOH/H2O (1:1) and n-hexane. The water layer was filtered to obtain a filtrate and a water-insoluble portion. This filtrate (55.5 g) was subjected to column chromatography on Diaion HP-20 (10×60 cm) using increasing concentrations of MeOH in H₂O as the eluent to obtain ten fractions (ACEW 1-10). Compounds II-11 (2.6 mg) and II-12 (2.2 mg) were obtained from fraction ACEW 1 by a silica gel column chromatography using benzene-CHCl₃ (9:1) as the eluent. Fraction ACEW 8 was rechromatographed on a silica gel column using CHCl₃-Me₂CO (25:1) as the eluent and purified further by preparative TLC (silica gel, i-Pr₂O-Me₂CO, 15:1) to obtain compounds II-7 (40.0 mg), II-4 (2.7 mg), II-5 (2.0 mg), and II-6 (2.5 mg). ACWE 10 was separated on a silica gel column using i-Pr₂O-MeOH (6:1) as the eluent to afford four subfractions (ACEW10-1-10-4). Compounds II-2 (10.0 mg), II-1 (2.0 mg), II-10 (3.2 mg), II-9 (10.2 mg), and II-8 (30.0 mg) were obtained from subfraction ACEW10-1 using preparative TLC (silica gel, n-hexane-Me2CO, 15:1). Compounds II-13 (7.0 mg), II-14 (6.1 mg), and II-15 (3.5 mg) were isolated from subfraction ACEW10-3 by column chromatography over silica gel using n-hexane-EtOAc (1:1) as the eluent. Subfraction ACEW10-4 was chromatographed on a silica gel column using n-hexane-EtOAc (1:1.5) as the eluent to yield compound II-3 (3.0 mg).

The n-hexane layer (9.3 g) was chromatographed on silica gel and eluted with EtOAc in n-hexane (0-100% of EtOAc, gradient) to obtain ten fractions. Fraction 4 was chromatographed repeatedly on a silica gel column using n-hexane-Me₂CO (19:1) as the eluent to yield compounds II-23 (3.0 mg), II-24 (6.0 mg), II-25 (4.5 mg), II-38 (3.0 mg), II-34 (22.0 mg), II-35 (90.2 mg), II-36 (22.1 mg), and II-37 (16.5 mg). Compound II-37 (41.1 mg) was also obtained in the same way from fraction 8. The water-insoluble portion (89.5 g) was chromatographed on a silica gel column using CHCl₃-MeOH mixtures of increasing polarity for elution to obtain ten fractions (WI-1-WI-10). Compounds II-16 (2.2 mg), II-20 (2.0 mg), II-21 (14.2 mg), II-24 (1.0 mg), II-29 (1.29 g), II-30 (53.8 mg), and II-36 (62.2 mg) were obtained from a combined fraction (fractions WI-1 and WI-2) by silica gel column chromatography with gradient elution (CHCl₃-Me₂CO, 39:1 to 14:1). Fraction WI-3 was separated on a silica gel column using i-Pr₂O-MeOH (19:1) as the eluent to yield compounds II-26 (141.5 mg), II-33 (11.0 mg), II-31 (122.9 mg), and II-27 (53.0 mg). Fraction WI-4 was chromatographed on a silica gel column with i-Pr₂O-MeOH (12:1) to give compounds 22 (11.3 mg), 33 (38.0 mg), 31 (708.0 mg), and II-27 (66.5 mg). Fractions WI-5-WI-7 were combined and rechromatographed on a silica gel column with CHCl₃-MeOH (6:1) as the mobile phase to afford compounds II-17 (5.0 mg), II-19 (2.2 mg), II-18 (3.8 mg), II-22 (3.4 mg), and II-28 (2.10 g). Compound II-32 (1.16 g) was isolated from a combined fraction (fractions WI-8 and WI-9) by silica gel column chromatography using i-Pr₂O-MeOH (4:1) as the eluent.

The obtained compounds II-1-II-38 are analyzed with the same methods and instruments as those used in Embodiment 1.

### Compound II-1

The compound II-1 was isolated as a pale yellow oil, and the analysis data thereof are listed as follow.

UV (MeOH) λₘₐₓ (log ε) 214 (3.44), 275 (2.63), 315 (2.94) nm; IR (KBr) νₘₐₓ 2925, 2854, 1663, 1610, 1475, 1446, 1381, 1277, 1212, 1054 cm⁻¹; ¹H NMR (CDCl₃ 400 MHz) δ_{H} 5.98 (2H, s, OCH2O), 4.02 (3H, s, OMe-6), 3.88 (3H, s, OMe-5), 2.45 (3H, s, 4′), 2.31 (3H, s, Me-4); ¹³C NMR (CDCl₃, 100 MHz) δ_{C} 184.8 (C-3′), 142.5 (C-2), 142.0 (C-6), 137.5 (C-5), 136.2 (C-1), 131.3 (C-4), 106.6 (C-3), 102.2 (OCH2O), 96.0 (C-2′), 87.6 (C-1′), 60.7 (OMe-6), 60.5 (OMe-6), 33.2 (C-4′), 14.4 (Me-4); ESIMS m/z 285 [M+Na]⁺; HRESIMS m/z 285.0740 (calculated for C₁₄H₁₄O₅Na, 285.0739).

These data helped to establish the structure of the compound II-1, and the result showed that the structure of the compound II-1 is represented by the following formula (II-1):

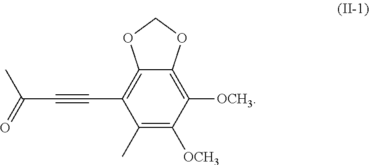 [SMILES: COc1c(C)c(C#CC(C)=O)c2c(c1OC)OCO2]

### Compound II-2

The compound II-2 was isolated as a pale yellow oil, and the analysis data thereof are listed as follow.

UV (MeOH) λₘₐₓ (log ε) 215 (4.38), 254 (3.78), 287 (4.04) nm; IR (KBr) νₘₐₓ 2943, 2781, 1611, 1473, 1449, 1389, 1274, 1207, 1050 cm⁻¹; ¹H NMR (CDCl₃, 400 MHz) δ_{H} 5.36 (1H, br s, H-5′ b), 5.26 (1H, br s, H-5′ a), 5.92 (2H, s, OCH2O), 3.97 (3H, s, OMe-6), 3.85 (3H, s, OMe-5), 2.26 (3H, s, Me-4), 2.00 (3H, s, Me-3′); ¹³C NMR (CDCl₃, 100 MHz) δ_{C} 139.8 (C-6), 139.4 (C-1), 137.1 (C-5), 136.2 (C-2), 127.8 (C-4), 127.2 (C-3′), 120.9 (C-5′), 109.8 (C-3), 101.4 (OCH2O), 97.5 (C-2′), 83.5 (C-1′), 60.3 (OMe-6), 59.9 (OMe-5), 23.5 (Me-4), 13.8 (Me-3′); ESIMS m/z 283 [M+Na]; HRESIMS m/z 283.0944 (calculated for C₁₅H₁₆O₄Na, 283.0946).

These data helped to establish the structure of the compound II-2, and the result showed that the structure of the compound II-2 is represented by the following formula (II-2):

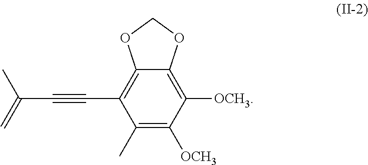 [SMILES: C=C(C)C#Cc1c(C)c(OC)c(OC)c2c1OCO2]

### Compound II-3

The compound II-3 was isolated as colorless oil, and the analysis data thereof are listed as follow.

UV (MeOH) λₘₐₓ (log ε) 220 (3.69), 263 (3.36), 320 (2.95) nm; IR (KBr) νₘₐₓ 2920, 2851, 1699, 1629, 1503, 1437, 1201, 1097 cm⁻¹; ¹H NMR (CDCl₃, 300 MHz) δ_{H} 6.90 (1H, s, H-6), 6.04 (2H, s, OCH2O), 4.10 (3H, s, OMe-4), 3.89 (3H, s, COOCH3), 3.85 (3H, s, OMe-5); ¹³C NMR (CDCl₃, 75 MHz) δ_{C} 164.9 (COOCH3), 146.4 (C-5), 144.8 (C-2), 137.7 (C-4), 137.5 (C-3) 104.8 (C-1), 104.3 (C-6), 102.1 (OCH2O), 60.2 (OMe-4), 56.7 (OMe-5), 52.0 (COOCH3); ESIMS m/z 263 [M+Na]⁺; HRESIMS m/z 263.0534 (calculated for C₁₁H₁₂O₆Na, 263.0532).

These data helped to establish the structure of the compound II-3, and the result showed that the structure of the compound II-3 is represented by the following formula (II-3):

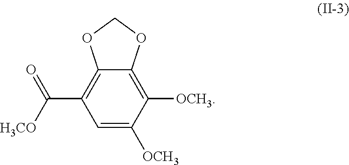 [SMILES: COC(=O)c1cc(OC)c(OC)c2c1OCO2]

### Compound II-4

The compound II-4 was isolated as white powder, and the analysis data thereof are listed as follow.

mp 73-74° C.; UV (MeOH) λₘₐₓ (loge) 207 (4.80), 279 (3.39) nm; IR (KBr) νₘₐₓ 2939, 2892, 1619, 1497, 1448, 1427, 1254, 1232, 1119, 1085, 1057, 1024, 956 cm⁻¹; ¹H NMR (CDCl₃ 500 MHz) δ_{H} 2.03 (3H, s, CH3-1), 2.06 (3H, s, CH3-1′), 3.82 (3H, s, OCH3-5), 3.88 (3H, s, OCH3-2′), 3.93 (3H, s, OCH3-2), 5.92 (1H, s, H-6′), 5.94 (2H, s, OCH2O-3,4), 5.98 (2H, s, OCH2O-3′,4′); ¹³C NMR (CDCl₃, 125 MHz) δ_{C} 9.3 (CH3-1), 15.8 (CH3-1′), 59.8 (OCH3-2′), 60.0 (OCH3-2), 60.6 (OCH3-5), 101.4 (OCH2O-3,4), 101.6 (OCH2O-3′,4′), 109.5 (C-6′), 117.6 (C-1), 123.6 (C-1′), 133.0 (C-5), 134.3 (C-3′), 135.6 (C-4), 136.7 (C-2′), 136.8 (C-2), 137.25 (C-5′), 137.29 (C-3), 138.7 (C-4′), 139.5 (C-6); ESIMS m/z 399 [M+Na]⁺; HRESIMS m/z 399.1052 (calculated for C₁₉H₂₀O₈Na, 399.1056).

These data helped to establish the structure of the compound II-4, and the result showed that the structure of the compound II-4 is represented by the following formula (II-4):

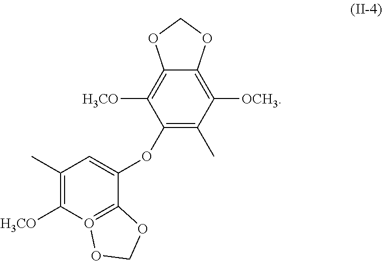

### Compound II-5

The compound II-5 was isolated as colorless oil, and the analysis data thereof are listed as follow.

UV (MeOH) λₘₐₓ (loge) 208 (4.91), 283 (3.80) nm; IR (KBr) νₘₐₓ 3526, 2928, 2859, 1713, 1492, 1460, 1261, 1035 cm⁻¹; ¹H NMR (CDCl₃, 500 MHz) δ_{H} 1.85 (6H, s, CH3-1,1′), 3.93 (6H, s, OCH3-2,2′), 6.02 (4H, s, OCH2O-3,4; 3′,4′); ¹³C NMR (CDCl₃, 125 MHz) δ_{C} 12.6 (CH3-1,1′), 60.1 (OCH3-2,2′), 101.8 (OCH2O-3,4; 3′,4′), 114.5 (C-6,6′), 123.8 (C-1,1′), 133.3 (C-5,5′), 133.6 (C-4,4′ or C-3, C-3′), 136.4 (C-2,2′), 139.1 (C-4,4′ or C-3, C-3′); ESIMS m/z 385 [M+Na]⁺; HRESIMS m/z 385.0897 (calculated for C₁₈H₁₈O₈Na, 385.0899).

These data helped to establish the structure of the compound II-5, and the result showed that the structure of the compound II-5 is represented by the following formula (II-5):

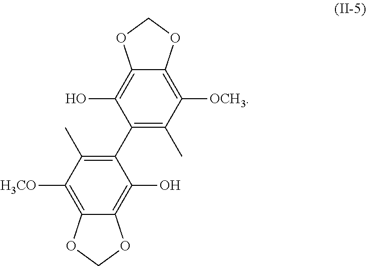 [SMILES: COc1c(C)c(-c2c(C)c(OC)c3c(c2O)OCO3)c(O)c2c1OCO2]

### Compounds II-6 to II-38

Other compounds obtained from Embodiment 2 are known compounds, including seven benzenoids, three lignans, and twenty-three triterpenoids, which were identified by the comparison of their physical and spectroscopic data with those of corresponding authentic samples. The seven benzenoids are 2,5-dimethoxy-3,4-methylenedioxybenzoate (II-6), 2,2′,5,5′-tetra-methoxy-3,4,3′,4′-bi-methylenedioxy-6,6′-dimethylbiphenyl (II-7), 4,7-dimethoxy-5-methyl-1,3-benzodioxole (II-8), antrocamphin A and B (II-9 and II-10), syringic acid (II-11), 3,4,5,-trimethoxybenzoic acid (II-12). The three lignans are 4-hydroxysesamin (II-13), (+) sesamin (II-14), and aptosimon (II-15). In addition, the twenty-three triterpenoids are camphoratins A-J (II-16-II-25), zhankuic acids A-C (II-26-II-28), zhankuic acid A methyl ester (II-29), antcin A (II-30), antcin C (II-31), antcin K (II-32), methyl antcinate H (II-33), eburicol (II-34), ergosterol D (II-35), methyl 4α-methylergost-8,24(28)-dien-3,11-dion-26-oate (II-36), ergosterol peroxide (II-37), and ergosta-2,4,8 (14),22-trtraen-3-one (II-38).

### Cytotoxicity Assay

Compounds II-7-II-9, II-13, II-14, II-20, II-21, II-25-II-33, and II-36 were assayed for cytotoxic activity against Doay (human medulloblastoma), Hep2 (human laryngeal carcinoma), MCF-7 (human breast adenocarcinoma), and Hela (human cervical epitheloid carcinoma) cell lines, using a MTT assay method. The assay procedure was carried out as previously described (Shen, Y. C.; Wang, S. S.; Pan, Y. L.; Lo, K. L.; Chakraborty, R.; Chien, C. T.; Kuo, Y. H.; Lin, Y. C. J. Nat. Prod. 2002, 65, 1848-1852.) and mitomycin was used as positive control with ED₅₀ values of 0.12, 0.14, 0.11, and 0.15 μg/mL (Doay, Hep2, MCF-7, and Hela, respectively).

The results of the cytotoxicity assay are shown in the following Table 6.

TABLE 6
Cytotoxicity data of compounds II-7-II-9, II-13, II-14, II-20,
II-21, II-25-II-33, and II-36

cell lines ED₅₀ (μg/mL)

| compound | Daoy | Hep2 | MCF-7 | Hela |
|---|---|---|---|---|
| II-9 | 5.9 | 10.5 | 3.4 | 6.9 |
| II-20 | 5.2 | 7.0 | 6.6 | 9.0 |
| II-21 | 4.4 | 3.0 | 7.9 | 8.9 |
| II-25 | —^{a} | — | 8.7 | 11.3 |
| II-26 | — | 16.6 | — | — |
| II-30 | 13.2 | — | 13.3 | — |
| Mitomycin C | 0.1 | 0.1 | 0.1 | 0.2 |

^{a}ED50 > 20 μg/mL.
^{b}Compounds II-7 and II-8, II-13 and II-14, II-27-II-29, II-31-II-33, and II-36 were inactive for all cell lines with ED₅₀ > 20 μg/mL.

As shown in Table 6, the compounds II-9 and II-21 showed significant cytotoxicity against MCF-7 and Hep2 cell lines with ED₅₀ values of 3.4 and 3.0 μg/mL, respectively. The other tested compounds were found to be not active against the above cancer cell lines.

In addition, the anti-inflammatory potentials of compounds II-2, II-7-II-9, II-17, II-21, and II-34-II-37 were evaluated by examining their effects on LPS-induced iNOS-dependent NO production and NOX-dependent ROS production in murine microglial cells (BV2) and peripheral human neutrophils (PMN), by the same method described in Embodiment 1. The results of these assays are listed in the following Table 7.

TABLE 7
Summary of the effects of compounds II-2, II-7-II-9, II-17, II-21,
and II-24-II-37 on NADPH oxidase (NOX) activity^{a} in murine microglial
cells (BV2) and peripheral human neutrophils (PMN) and nitric oxide
synthase (NOS) activity^{b} in murine microglial cells

| | IC₅₀ (μM) in NOX | IC₅₀ (μM) in NOX | |
|---|---|---|---|
| | activity from BV2 | fMLP-induced NOX | |
| | cell lysate | activation in PMN | IC₅₀ (μM) in NOS |
| II-2 | ND | 14.4 ± 4.9* | 12.1 ± 0* |
| II-7 | ND | 15.5 ± 3.3* | 16.2 ± 1.4* |
| II-8 | ND | 19.9 ± 3.0* | 29.1 ± 4.4* |
| II-9 | 50.1 ± 3.3* | 15.1 ± 4.1* | 7.2 ± 1.0* |
| II-17 | ND | 32.1 ± 3.5* | 15.7 ± 0.9* |
| II-21 | ND | 11.2 ± 2.3* | 2.5 ± 0.6* |
| II-24 | ND | 17.5 ± 3.9* | 12.7 ± 2.2* |
| II-25 | ND | 15.8 ± 4.0* | 1.6 ± 0.6* |
| II-26 | ND | 22.1 ± 6.7* | 3.6 ± 0.8* |
| II-27 | ND | ND | 9.6 ± 0.7* |
| II-28 | 40.3 ± 3.5* | ND | 16.2 ± 0.9* |
| II-29 | ND | 8.4 ± 2.1* | 0.6 ± 0.3* |
| II-30 | 45.9 ± 7.9* | 29.2 ± 6.7* | 4.1 ± 0.5* |
| II-31 | ND | 22.6 ± 3.3* | 4.2 ± 1.2* |
| II-32 | ND | 47.2 ± 8.4* | ND |
| II-33 | 16.0 ± 8.1* | 18.1 ± 5.9* | 2.5 ± 0.3* |
| II-34 | ND | 21.9 ± 6.3* | 22.3 ± 2.9* |
| II-35 | ND | 27.9 ± 5.6* | 30.6 ± 0.8* |
| II-36 | ND | 16.2 ± 4.3* | 1.5 ± 0.7* |
| II-37 | ND | 20.3 ± 6.4* | 6.3 ± 1.8* |
| DPI | 0.4 ± 0.2 | 0.3 ± 0.1 | — |
| L-NAME | — | — | 25.8 ± 2.5 |

^{a}NADPH oxidase (NOX) activity were measured as reactive oxygen species production by triggering with NADPH (200 μM) or fMLP (2 μM) in the presence 1-50 μM of test drugs in BV2 cell lysate or peripheral human neutrophils (PMN). Diphenyleneiodonium (DPI, a NOX inhibitor) was included as a positive control for NOX inhibition.
^{b}NO production was measured in the presence of 1-50 μM of test drugs.
L-NAME (a non-selective NOS inhibitor) was included a positive control.
Data were calculated as 50% inhibitory concentration (IC₅₀) and expressed as the mean ± S.E.M. from 3-6 experiments performed on different days using BV2 cell lysate or PMN from different passages or donors.
ND: values not detectable.
“—”: samples not tested.
*P < 0.05 as compared with relative positive control.

Triterpenoids II-21, II-25 and II-26, II-29-II-31, II-33, and II-36 significantly inhibited NOS activity (IC₅₀<5 μM) with IC₅₀ values of 2.5, 1.6, 3.6, 0.6, 4.1, 4.2, 2.5, and 1.5 μM, respectively. These compounds were more potent than L-NAME (IC₅₀ 25.8 μM), a nonspecific NOS inhibitor, at inhibiting LPS-induced NO production. The other compounds, except for II-8 and II-35, also effectively inhibited NOS activity with IC₅₀ values ranging from 6.3 to 22.3 μM.

In addition, the data for evaluating the effects of these compounds on NOX activity in lysates of microglial cells and PMN suggest none of the tested compounds were potent inhibitors of NOX in lysates of microglial cells and PMN, relative to the specific NOX inhibitor DPI (IC₅₀ 0.4 and 0.3 μM, respectively), as shown in Table 7.

Furthermore, the free radical-scavenging capacities of these compounds were examined in a cell-free 1,1-diphenyl-2-picrylhydrazyl (DPPH) solution. However, none of these tested compounds showed considerable free radical-scavenging activity. Therefore, the results revealed that the triterpenoids II-21, II-25 and II-26, II-29-II-31, II-33, and II-36 have potent NO-reducing activity in microglial cells.

Although the present invention has been explained in relation to its preferred embodiment, it is to be understood that many other possible modifications and variations can be made without departing from the spirit and scope of the invention as hereinafter claimed.

What is claimed is:

1. A benzenoid derivative represented by the following formula (II): 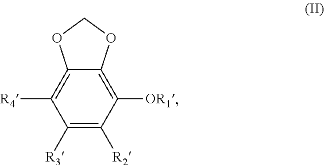
wherein the benzenoid derivative is a compound represented by the following formula (II-1), (II-2), (II-3), (II-4), or (II-5):

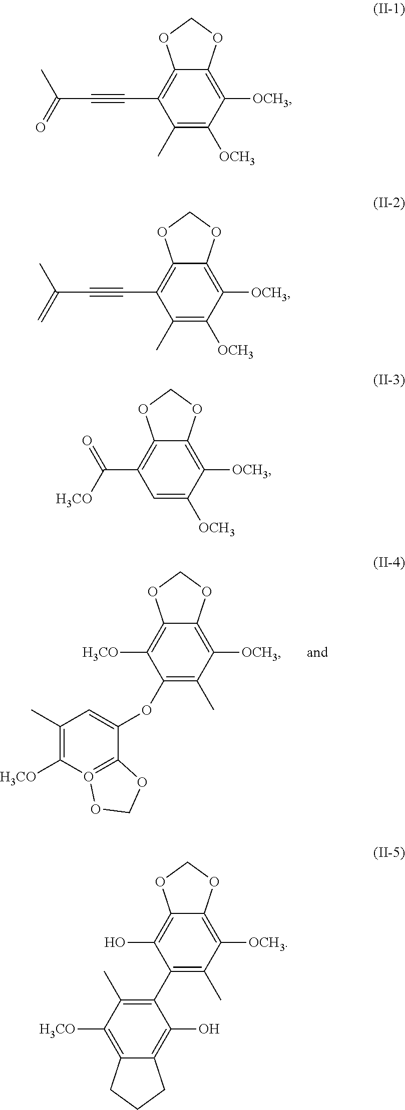

2. A pharmaceutical composition comprising an effective amount of a benzenoid derivative represented by the following formula (II) 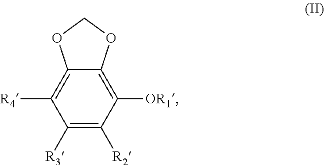
and a pharmaceutically acceptable carrier, wherein the benzenoid derivative is a compound represented by the following formula (II-1), (II-2), (II-3), (II-4), or (II-5):

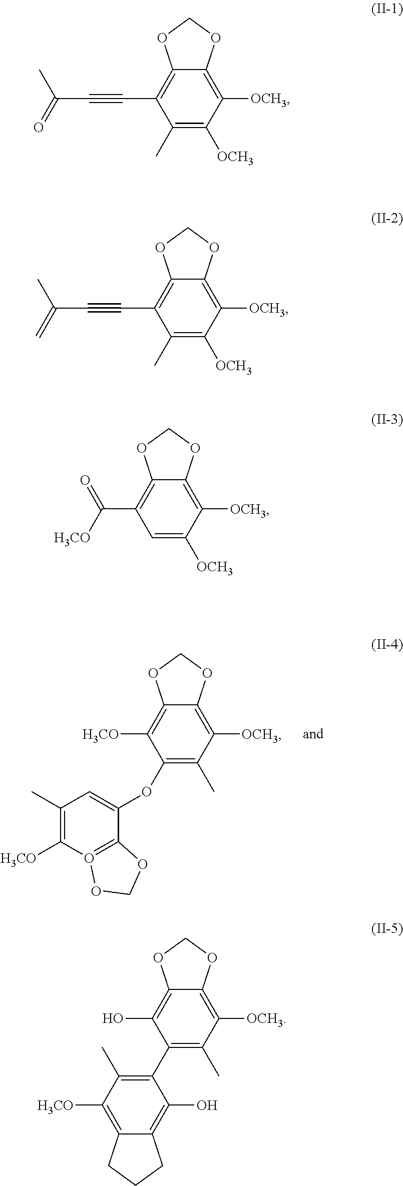

3. The pharmaceutical composition as claimed in claim 2, which has anti-inflammation or cancer cell cytotoxic activity.
